(19) United States
(12) Patent Application Publication
(10) Pub. No.: US 2012/0213873 A1
(43) Pub. Date: Aug. 23, 2012

(54) Miracle cream

(76) Inventor: Gerald De Marco, Stoneham, MA (US)

(21) Appl. No.: 12/932,133

(22) Filed: Feb. 18, 2011

(51) Int. Cl. A61K 36/886; A61P 17/00; A61Q 19/08; A61Q 19/00; A61K 8/97; A61P 17/02

(52) U.S. Cl. 424/744

(57) ABSTRACT

This composition stimulates collagen production; specifically the composition is able to promote collagen production in human dermis.

This invention promotes collagen production having Barbadiensis Miller Aloe Vera, iron, ascorbic acid as active ingredients.

The way to promote collagen production is applying this active ingredients, Barbadiensis Miller aloe vera, iron and ascorbic acid are contained in the composition that is applied directly to the skin twice a day

MIRACLE CREAM

| REFERENCE | | |
|---|---|---|
| 7,700,083 | May 20, 2010 | Meeh an Kevin |
| 7,807,625 | Oct. 5, 2010 | Mayenski, et al |
| 7,645,796 | Jan. 12, 2010 | Murakami et al |
| 7,557,093 | Jul. 7, 2009 | Shinohara, et al |
| 7,396,912 | Jul. 8, 2008 | Hsiao et al |
| 7,306,810 | Dec. 11, 2007 | Spencer David, M |
| RE39043 | Mar. 28, 2006 | Nakayama et al |
| 6,896,889 | May 24, 2005 | Chevalier, et al |

### THE BACKGROUND OF THE INVENTION

80% of the dermis of the skin is collagen; its functions are support the skin structure, retain moisture and provide an environment for cell survival.

During the aging process the collagen a mount diminishes 50% at the dermis level at 40 years old, the elasticity of the skin is diminished, the skin becomes thinner, diminish oil production by sebaceous glands, diminish the recovery capacity of the skin after an injury, the natural texture is compromised, the skin is less hydrated.

Consequently clinical signs of the aging process appears, fine wrinkles, deep wrinkles, thin skin, dry skin, easy to crack skin due to a diminish elasticity and thickness, of the skin, deeper expression marks, sagging skin

All this signs increase with age and exposition of the skin to ultra violet rays (sunbath, tanning booth), smoking, alcohol intake, diminished sleep time, stress, unhealthy diet, and facial expressions which generates expression marks, making worst the appearance of an already wrinkle face.

Elements like a-hydroxyl acids, b-hydroxyl acids and retinoid are applied to the skin to fight against the aging signs, these elements act on wrinkles by eliminating dead skin cells and accelerating the renewal process but they don't act on the real cause of the winkle formation.

Other elements containing collagen, they are applied directly to the skin, assuming that they will get absorbed and the skin will get a greater amount of collagen afterwards, collagen is a protein, proteins are too big of a molecule to be absorb by the skin, consequently this type of composition it does n of improve wrinkles.

Another elements contain tensing elements such as milk, soya protein extracts, but substances from natural sources like cow milk has the risk of producing bovine spongiform encephalopathy, also the tensing effects are marginal and at times not reproducible, besides the real tensing effect is given by collagen.

### SUMMARY OF THE INVENTION

The inventor discovered that increasing the amount of collagen in a natural way, meaning being fabricated by the dermis itself, it is the most efficient way to increase the collagen amount at the skin level.

This process is done stimulating collagen formation with active elements which are Barbadiensis Miller Aloe Vera, Iron and Ascorbic Acid which are the main components of the composition invented, with the natural support of the nutrients, glycine, proline, hydrxyproline, a-ketoglutarate and Oxygen delivery by the blood supply regularly.

This composition develops a full collagen skin, a thicker, more elastic, non-easily cracking skin and the healing and clearing capacity of the skin is improved.

Consequently wrinkles and expression marks formation are diminished, there is an improvement of the wrinkles and expression marks already present, less scar formation after an injury is encounter when the composition is applied immediately after the injury, acne old scars improved, skin dark discolorations improved, the healing process of thermal injury and wounds are improved.

### DETAIL DESCRIPTION OF THE INVENTION

The collagen formation at dermis level is stimulated by Barbadiensis Miler Aloe Vera, its formation and production is possible due to the local presence of Iron and Ascorbic Acid and supported by nutrient elements, glycine, proline, hydroxyproline, a-ketoglutarate and Oxygen naturally delivery by the blood supply, so there is no need to locally applied these elements to the skin, proline gets hydroxilated to hydroxyproline inside the organism.

Collagen is a protein produced by the fibroblasts of the dermis consequently an increase production of collagen stimulated by the composition will create a skin that is thicker, more elastic, with better texture; also the healing and clearing capacity of the skin will improve.

The composition contains Barbadiensis Miller Aloe Vera in the amount of 50 to 70% of the total weight of the composition, Ascorbic Acid in the a mount of 100 mg, 0.1% of the total weight of the composition, Ferrous Sulfate which provides 3.48 mg of iron, 0.00348% of the total weight of the composition, humectants elements which are water in the amount of 25% of the total weight of the composition, petrolatum 3.0%, polysorbato 20 3.0%, dimeticone 2.0%, tocopheryacetate 1.0%, peg 5 soy sterol 5.0%, cetyl alcohol 5.0%, stearic acid 2.0% , glycerin 3.0%, phenoxyethanol 1.0%. The composition contains preservatives DMDM hydantoin 0.05%, tetrasodium EDTA 0.05%

The amount of the humectants portion of the composition is 30 to 50% of the total weight of the composition.

The total weight of the composition is 100 grams.

First gets mixture the humectants portion plus the preservatives with the barbadiensis Miller Aloe Vera portion of the composition at 300 RPM for 10 minutes then Ascorbic Acid is added and get mixture at 300 RPM for 5 minutes finally Ferrous Sulfate is added and gets mixture at 300 RPM for 5 minutes

Consequently fire wrinkles disappear after 3 months of application of the composition, moderate wrinkles (2 mm deep) improve to 1 mm deep after 6 months of application of the composition, expression marks of moderate degree, (2 mm deep) improves to 1 mm deep after 6 months of application of the composition, mild hypotrophic scar, 1 mm deep disappear after 3 months of application, moderate hypotrophic, 2 mm deep improve to 1 mm deep after 6 months of application, severe hypotrophic 3 mm deep improve to 1 mm deep after 12 months of application, fresh skin cut 3 mm deep, 3 mm wide immediately after applying the composition for 3 months no scar formation is found and the cut is totally heal with normal looking skin formation. The composition clears the skin and improves dark discoloration.

### Example 1

Babisiensis Mille Aloe Vera 50 grams, water 25 grams, petrolatum 3 gr, polysorbate 2.0 gr, dimeticone 2.0 gr, tocopheryl acetate 1.0 gr, peg 5 soy sterol 5.0 gr, cetyl alcohol 5.0 gr, stearic acid 2.0 grs, glycerin 3.0 gr, phenoxyethanol 1.0 gr, DMDM hydantoin 0.05 gr, tetrasodium EDTA 0.05 gr.

### Example 2

Babisiensis Miller Aloe Vera 60 gr. Water 15 gr, petrolatum 3.0 gr. Isopropyl palmitato 3.0 gr. Sorbitan oleato 2.0 gr. Tocopheryl acetate 1.0 gr. Triethanolamine 5.0 gr. Cetyl alcohol 5.0 gr. Carbomer 2.0 grs. Glycerin 3.0 gr. Phenoxyethanol 1.0 gr. DMDM hydantoin, tetra sodium EDTA 0.05 gr, iron 0.00348 gr, ascorbic acid 0.1 gr.

### Test Example 1

The example 1 composition was given to 50 y/o males, twice a day.

| | 3 | 6 | 12 |
|---|---|---|---|
| SIGNS | MONTHS | MONTHS | MONTHS |
| Fine wrinkles | disappear | | |
| Wrinkles (2 mm deep) | | 1 mm deep | |
| Expression marks (2 mm deep) | | 1 mm deep | |
| Hypotrophic acne scar | disappear | | |
| (1 mm deep) | | | |
| Hypotrophic acne scar | | 1 mm deep | |
| (2 mm deep) | | | |
| Hypotrophic acne scar | | | 1 mm deep |
| (3 mm deep) | | | |
| Skin cut(3 mm deep/ | no scar, | | |
| 3 mm wide) | healed | | |

Starting at 50 y/o with continuous application of example 1 composition for 4 years a delay presentation of fine wrinkles and mild expression marks (1 mm deep) was observed.

### Test Example 2

The example 2 composition was given to 50 y/o females

| | 3 | 6 | 12 |
|---|---|---|---|
| SIGNS | MONTHS | MONTHS | MONTHS |
| Fine wrinkles | disappear | | |
| Wrinkles (2 mm deep) | | 1 mm deep | |
| Expression marks (2 mm deep) | | 1 mm deep | |
| Hypotrophic acne scar | disappear | | |
| (1 mm deep) | | | |
| Hypotrophic acne scar | | 1 mm deep | |
| (2 mm deep) | | | |
| Hypotrophic acne scar | | | 1 mm deep |
| (3 mm deep) | | | |
| Skin cut (3 mm deep/ | no scar, | | |
| 3 mm wide) | healed | | |

1. A composition generates collagen formation at the dermis level, by natural stimulation by Barbadiensis Miller Aloe Vera, securing the collagen formation by the necessary elements needed in collagen creation which are iron, ascorbic acid and nutrients glycine, proline, hydroxyproline, a-ketoglutarate and oxygen which are delivery by the blood supply.

2. A method of promoting collagen production according to claim 1 wherein at least one active material amount ranging from 50 to 70% of the weight based on the total weight of the composition, the one active material is Barbadiensis Miller Aloe Vera

3. A method of promoting collagen production according to claim 1, wherein at least a second active material which is Iron in the amount of 3.48 mg. Essential active element to generate collagen formation

4. A method of promoting collagen production according claim1, wherein at least having a third active material which is Ascorbic Acid in the amount of 100 mg. Ascorbic Acid is an essential element working with Babisiensis Miller Aloe Vera and Iron to generate collagen formation

5. A method promoting collagen production according to claim 1, wherein gives more elasticity to the skin, elasticity generated by an increase amount of collagen at skin level

6. A method of promoting collagen production according claim 1, wherein improves mild, moderate and severe hypotrophic acne scars by collagen formation.

7. A method promoting collagen formation according to claim 1, wherein improves wrinkles already present by adding more collagen and elasticity to the area of the skin where wrinkles are located.

8. A method promoting collagen formation according claim 1, wherein improves expression marks already present by adding more collagen and elasticity to the area of the skin where the expression marks are located.

9. A method of promoting collagen formation according claim 1, wherein delays the wrinkle process formation because the skin has more capacity to resist the creation of wrinkles due to an increase amount of collagen and elasticity in the area of the skin where the composition was applied.

10. A method of promoting collagen formation according claim 1, wherein delays the expression mark formation process because the skin has more capacity to resist the creation of expression marks due to an increase amount of collagen and elasticity in the area of the skin where the composition is applied.

11. A method for promoting collagen formation according to claim 1, wherein creates a thicker skin due to increase wideness of the dermis due to greater deposit of collagen at dermis level.

12. A method for promoting collagen formation according to claim 1, diminish scar formation due to an injury if applied immediately after the injury.

13. A method for promoting collagen formation according claim 1, wherein improves the texture of the skin.

14. A method for promoting collagen formation according claim 1, wherein creates a younger skin.

15. A method for promoting collagen formation according claim 1, wherein creates a better looking skin.

16. A method for promoting collagen formation according claim 1, helps to recover from thermal injury or wound due to its increase healing capacity.

17. A composition having humectants elements in the amount of 30 to 50% of the weight based on the total weight of the composition.

18. According to claim 16, the humectants elements are water, polysorbate 20, dimeticone, tocophery acetate, peg-5 soy sterol, cetyl alcohol, stearic alcohol, glycerin, phenoxyethanol, they hydrate the skin and the hydration is preserved at skin level due to an increase collagen level.

19. The composition according claim 2, has Babadiensis Miller Aloe Vera which makes a more clear skin.

20. The composition according claim 2, has Barbadiensis Miller Aloe Vera which improves dark discoloration of the skin.
